(19) United States
(12) Patent Application Publication
(10) Pub. No.: US 2004/0267456 A1
(43) Pub. Date: Dec. 30, 2004

(54) Method and computer program product for drug discovery using weighted grand canonical metropolis Monte Carlo sampling

(76) Inventors: Stephan Brunner, Preverenges (CH); Charles Karney, Princeton, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/794,181

(22) Filed: Mar. 8, 2004

Related U.S. Application Data
(60) Provisional application No. 60/482,774, filed on Jun. 27, 2003.
(60) Provisional application No. 60/509,272, filed on Oct. 8, 2003.
(60) Provisional application No. 60/509,543, filed on Oct. 9, 2003.
(60) Provisional application No. 60/531,687, filed on Dec. 23, 2003.

(51) Int. Cl. G06G 7/48; G06G 7/58; G06F 19/00; G01N 33/48; G01N 33/50

(52) U.S. Cl. 702/19; 703/11

(57) ABSTRACT

A method and computer program product for modeling a system that includes a protein and a plurality of different fragment types in order to identify drug leads is presented. The basis of the method is a weighted Metropolis Monte Carlo approach for sampling the grand canonical ensemble. This method distinguishes itself from an energy minimization approach in that it provides fragment distributions which are consistent with thermal fluctuations at physiologically relevant temperatures. The weighted Metropolis Monte Carlo scheme performs a quasi-uniform sampling of all regions of interest on the protein, and, in this way, enables to resolve the wide range in densities of the thermodynamic distribution which could not be achieved by a non-weighted Metropolis scheme. Making use of the properties of the grand canonical ensemble, the affinity of fragments for different regions on the protein surface can be efficiently computed, using a so-called “simulated annealing of the chemical potential” process. A protein binding site is then identified as a region with high affinity for multiple fragments with a diverse set of physico-chemical properties. Within a binding site, assembly of fragments into drug leads is finally carried out based on binding affinity of the different fragments, on geometric proximity, and a variety of rules by which organic fragments may bond together.

This patent application claims the benefit of U.S. Provisional Patent Application No. 60/482,774 (filed Jun. 27, 2003), U.S. Provisional Patent Application No. 60/509,272 (filed Oct. 8, 2003), U.S. Provisional Patent Application No. 60/509,543 (filed Oct. 9, 2003), and U.S. Provisional Patent Application entitled “Method and Computer Program Product for Drug Discovery Using Weighted Grand Canonical Metropolis Monte Carlo Sampling,” serial number to be determined, SKGF Ref. 1866.0510000 (filed Dec. 23, 2003), all of which are incorporated herein by reference in their entireties.

### BACKGROUND OF THE INVENTION

1. Field of the Invention

The invention described herein relates to models for molecular interaction, and in particular the use of such models for drug discovery.

2. Related Art

In determining drug leads, it is often desirable to model a system that includes a protein and a set of small molecular fragments. Given the three dimensional structure of a target protein, usually obtained experimentally from x-ray crystallography, the basic interactions between the protein and the small fragments (typical average molecular weight of 150) are computed. This computation can be carried out by Monte Carlo (MC)-type modeling and analysis (usually implemented in software) for a large collection of organic fragments with diverse physico-chemical properties. For such a fragment-based approach, the number of considered fragments can in practice be in the hundreds to thousands. What are needed, therefore, are an efficient method and computer program product for modeling such a system of fragments for purposes of determining drug leads.

### SUMMARY OF THE INVENTION

The invention described herein includes a method and computer program product for modeling a system that comprises a protein and a plurality of fragments in order to identify drug leads. To analyze the interaction between a given type of fragment and a protein, the states of the fragment with respect to the protein are sampled from a thermodynamically relevant distribution. The underlying sampling algorithm is a Weighted Grand Canonical Metropolis Monte Carlo approach, referred to herein as WGCMMC.

The purpose of this weighted approach is to enable an essentially uniform numerical sampling of all states of interest of the fragment with respect to the protein, i.e. sampling deeper and shallower energy wells with the same thoroughness, while still avoiding the sampling of very unfavorable poses (typically resulting from steric clashes). The data is then appropriately re-weighted for the sampling to correctly represent the considered thermodynamic ensemble.

Saving a state of a system described by a grand canonical ensemble comprises saving the states of all fragments currently present in the system. In turn, saving a fragment state comprises storing its position, orientation, potential energy and weight. Note that in the framework of the grand canonical ensemble, the number of fragments in the system fluctuates from one system state to another.

By making use of this fragment data, binding modes can be identified and corresponding binding free energies estimated. The fact that the simulation system is considered in the framework of the grand canonical ensemble, instead of the canonical ensemble, enables through simulated annealing of the chemical potential an efficient estimation of the free energy of binding of a given fragment type for various binding modes on the protein surface. This binding data for the different fragment types can then in turn be used for identifying the relevant protein binding sites, and for assembling the different fragment types to obtain larger ligand molecules.

In practice, the weighting procedure is implemented by subdividing the sampling space with a grid. Each grid cell center x is assigned a local, numerical chemical potential field value Bₙᵤₘ,(x), which is adapted iteratively during the computation, based on preceding sampling statistics, so as to ultimately ensure an approximately uniform numerical sampling of fragment states at all regions of interest around the protein. Bₙᵤₘ is related to the energetic cost of inserting or removing a fragment from the numerical distribution in the cell centered at x, and the difference between its local value Bₙᵤₘ (x) and the actual physical chemical potential B of the system defines the weight w for each sampled fragment state.

Once the Bₙᵤₘ field has sufficiently converged, as a result of successive iterations, and the Markov chain associated with the Metropolis algorithm has equilibrated, the actual Monte Carlo sampling can be gathered. This is carried out by keeping the Bₙᵤₘ field fixed, and then periodically saving the state of the system along the Markov chain. The number of Markov steps interspacing the gathered states must naturally be sufficiently large to ensure proper decorrelation.

Further embodiments, features, and advantages of the present inventions, as well as the structure and operation of the various embodiments of the present invention, are described in detail below with reference to the accompanying drawings.

### DESCRIPTION OF THE FIGURES

FIG. 1 is a flowchart illustrating overall processing of an embodiment of the invention.

FIG. 2 is a flowchart illustrating the initial step of preparing a molecular model for the system to be analyzed.

FIG. 3 is a flowchart illustrating the modeling process at the systemic level for computing the fragment-protein interactions using a Weighted Grand Canonical Metropolis Monte Carlo (WGCMMC) approach, according to an embodiment of the invention.

FIG. 4 is a flowchart illustrating the convergence phase of the simulation system, according to an embodiment of the invention.

FIG. 5 is a flowchart illustrating the sampling phase of the simulation system, according to an embodiment of the invention.

FIG. 6 is a flowchart illustrating the process of identifying potential binding sites, according to an embodiment of the invention.

FIG. 7 is a flowchart illustrating the process of clumping fragments before assembly into drug leads, according to an embodiment of the invention.

FIG. 8 is a block diagram illustrating a computing platform on which a software embodiment of the invention can be stored and executed.

### DETAILED DESCRIPTION OF THE INVENTION

A preferred embodiment of the present invention is now described with reference to the figures, where like reference numbers indicate identical or functionally similar elements. Also in the figures, the left-most digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention. It will be apparent to a person skilled in the relevant art that this invention can also be employed in a variety of other devices and applications.

I. Overview

The invention described herein is a fragment-based approach for designing drug leads. For this purpose, Locus Pharmaceuticals, Inc., Blue Bell, Pa., developed the Locus Monte Carlo (LMC) code. The approach described herein makes use of a Weighted Grand Canonical Metropolis Monte Carlo (WGCMMC) algorithm for sampling fragment states around the target protein, of a given fragment type. This sampling data can then be directly used for estimating the free energy of binding for different binding modes of the given fragment type on the protein surface. This computation can be carried out simultaneously for hundreds to thousands of different fragment types on a computing platform consisting of multiple processors, such as a PC cluster. This approach mainly distinguishes itself from a similar process implemented by Mezei and Guamieri in their Metropolis Monte Carlo (MMC) code [Guamieri, F. and Mezei, M., J. Am. Chem. Soc. 118:8493-8494 (1996)], in that it removes fragment-fragment interactions.

During the Monte Carlo sampling, a set of attributes are saved for each rigid fragment instance, including the coordinates (x,y,z) of the fragment's center of mass, the unit quatemion q=(q₁, q₂, q₃, q₄) characterizing its orientation, and the potential energy of interaction E between the fragment and the protein.

This LMC data for the different fragment types can be analyzed for identifying potential binding sites using diagnostic tools such as the Locus Cluster Analysis (LCA) code and the Locus Binding Analysis (LBA) code (Locus Pharmaceuticals, Inc., Blue Bell, Pa.). These tools are based on the postulate that a protein binding site must be a localized high affinity region for a diverse collection of fragments, i.e. fragments with different physico-chemical properties. It is indeed assumed, that diverse interactions in a localized region are the necessary condition for ensuring the specificity of a binding site. If available, one naturally also makes use of experimental binding site data (e.g., co-crystal X-ray data and residue mutational analysis) in determining the final site within which the leads are designed.

Within the chosen binding site, fragments can be assembled into the actual candidate drug leads, usually composed of four to five fragments and thus having a molecular weight of the order of 600-800, using a software package such as the Locus Chemistry Design (LCD) software (Locus Pharmaceuticals, Inc., Blue Bell Pa.). Here again, use is made of the LMC fragment data in providing preferred fragment states—positions and orientations—with respect to the protein (also called fragment poses). Assembly of fragments is carried out based on geometric proximity, and using a variety of rules by which organic fragments may bond together. In somewhat more detail, two fragments can be assembled, if the relative positions of their atoms enable, within given tolerances, to establish a certain type of covalent bond, with specific bond lengths and angles. The most elementary example of bonding rule is of the form

—CH₂—H+H—CH₂— [symbol] —CH₂—CH₂

Other bonding rules, such as the fusing of methyl groups or merging of cyclic rings, for example, may also be considered.

Fragment-based computational approaches are well-known. One example is the Multiple Copy Simultaneous Search (MCSS) numerical tool presently commercialized by Accelrys, of San Diego, Calif., which derives from an original version developed by the group of Karplus, Harvard University, MA, [Miranker, A. and Kaprlus, M., Proteins: Struc. Func. Gen. 11:29-34 (1991); Caflish, A., et al., J. Med. Chem. 36:2142-2167 (1993); Joseph-McCarthy, D., et al., J. Am. Chem. Soc. 123:12758-12769 (2001)]. (These references are incorporated herein by reference in their entirety.)

What distinguishes the LMC approach from previous fragment-based methods is its ability to compute the actual thermodynamic fragment distributions around the protein, i.e. distributions consistent with thermal fluctuations at physiological temperatures. Information on the thermodynamic distribution is essential for computing free energies of binding, which, as presented further on, is the basic biologically relevant quantity for quantifying the binding affinity of a ligand.

Indeed, the MCSS approach for example is essentially based on an energy minimization procedure, providing fragment states corresponding to various local minima of the potential energy field representing the fragment-protein interaction. Such a procedure is computationally more expeditious than computing a thermodynamic ensemble of states, but is unable to provide information on entropic effects, essential for free energy estimates.

For computing the thermodynamic distributions, the LMC code package makes use of a Metropolis Monte Carlo approach [Metropolis, N., et al., J. Chem. Physics 21:1087-1092 (1953)] for sampling from a grand canonical ensemble of states [Adams, D. J., Molecular Physics 29:307-311 (1975); Mezei, M., Molecular Physics 61:565-582 (1987)]. (These references are incorporated herein by reference in their entirety.) In addition to exchanging just energy with a surrounding thermal bath, as in the case of a canonical ensemble, the system described by a grand canonical ensemble exchanges particles (or fragments in the case of LMC) with its surroundings as well. The energy cost associated with inserting/deleting a fragment from the system is determined by its chemical potential. By varying this chemical potential, so-called simulated annealing of the chemical potential, one may vary the average number of fragments in the simulation system. It is shown further on, that measuring the values of the chemical potential at which fragments leave various sites on the protein provides an estimate of the free energy of binding for the different binding modes over the protein surface.

The practicality of the simulated annealing procedure for estimating binding affinities was demonstrated by Guarnieri and Mezei for differentiating hydration propensities of different DNA grooves [Guarnieri, F. and Mezei, M., J. Am. Chem. Soc. 118:8493-8494 (1996)]. (This reference is incorporated herein by reference in its entirety.) These results were obtained with the Metropolis Monte Carlo (MMC) code developed by the group of Mezei, Mount Sinai School of Medicine, NY. For these simulations, the system was composed of a molecule fraction of DNA surrounded by a varying number of interacting water molecules.

In its original form, the LMC algorithm carried out a series of calculations similar to the MMC approach for each fragment-type of interest, i.e. simulations in which both the fragment—protein as well as all fragment—fragment interactions were considered. However, it has been acknowledged, that considering fragment-fragment interactions is actually detrimental to the physical interpretation of the simulation results for all fragments but water. Indeed, due to the high dilution of the solute molecules in actual biochemical relevant conditions, considering interactions between non-water fragments is not realistic. Furthermore, the drug leads assembled by LCD usually are composed of only one fragment of each type. Fragment-fragment interactions in the LMC simulation thus lead to undesirable correlation effects.

Finally, in the original MMC code, carrying out the simulated annealing of the chemical potential for computing the free energies of binding for a single fragment type required the data from multiple ensemble samplings at various B values, i.e. data from multiple simulations. In the absence of fragment-fragment interactions however, the data required for applying the simulated annealing procedure can be directly derived from the sampling of a single simulation. As will be shown further on, this simplification results from the ability of establishing the analytical dependence in B of the fragment density when fragment interactions are omitted. This fact naturally provides an opportunity for significant computational speedup.

It turns out however that the standard Metropolis Monte Carlo algorithm has difficulty in handling simulations where fragment-fragment interactions are removed. Indeed, the absence of fragment-fragment interactions leads to the possible overlap of fragments and thus to a broad range (typically orders of magnitude) of fragment densities between the higher and lower affinity binding sites on the protein, which the standard Metropolis Monte Carlo scheme has trouble in resolving. This problem has been overcome in the current implementation of LMC by developing a weighted Metropolis Monte Carlo approach.

The system in which fragment-fragment interactions have been removed can be referred to as being linear by reference to the linear properties of the partial differential equation (Liouville-type) that describes the time-evolution of the fragment density away from thermodynamic equilibrium.

II. Process

A. Formulation

First, the derivation of the single fragment density in the framework of the grand canonical ensemble is presented.

The potential energy of the system composed of N equivalent, rigid fragments is denoted U(Γ, N). In general, U includes both contributions from fragment-protein and fragment-fragment interactions. The configuration of the system is characterized by

Γ=(Y₁,Y₂, . . . , Y_{N}), (1)

where Yᵢ=(xᵢ, Ωᵢ) stands for the position xᵢ and orientation Ωᵢ of the rigid fragment i. In practice, the orientation Ωᵢ is conveniently represented by a unit quaternion q.

In the grand canonical ensemble, the probability that the system has N fragments in configuration Γ is given by

$$f ( Γ , N ) = \frac{1}{Q} \frac{1}{{V}^{N} {σ}^{N}} \frac{1}{N !} exp [ BN - β U ( Γ , N ) ] , \qquad ( 2 )$$

with the normalization factor given by the grand partition function

$$Q = {\sum }_{N = 0}^{∞} \frac{1}{N !} exp ( BN ) \int \frac{ⅆ {Y}^{N}}{{V}^{N} {σ}^{N}} exp [ - β U ( Γ , N ) ] . \qquad ( 3 )$$

Here V is the volume of the system, σ is the volume of orientation space, β=1/K_{B}T, T is the temperature, K_{B} the Boltzmann constant, and B is related to the excess chemical potential μ^{ex}, i.e. the energy cost in units of β⁻¹ for a particle to leave the system, according to the following relation:

B=βμ^{ex}+log<N>, (4)

where <N> is the average number of fragments in the system. The integral in Eq. (3) is taken over the whole configuration space (Vσ)^{N}.

Assuming no fragment-fragment interactions, the potential energy U of the system becomes:

$$U ( Γ , N ) = {\sum }_{i = 1}^{N} E ( {Y}_{i} ) , \qquad ( 5 )$$

where E(Y) is the energy of interaction of a single fragment of the considered type with the protein.

The grand partition function can then be written as

$$\begin{matrix}Q = {\sum }_{N = 0}^{∞} \frac{1}{N !} {( exp ( B ) \int \frac{ⅆ Y}{V σ} exp [ - β E ( Y ) ] )}^{N} = exp Z , \\ with & ( 6 ) \\ Z = exp ( B ) \int \frac{ⅆ Y}{V σ} exp [ - B E ( Y ) ] . & ( 7 )\end{matrix}$$

In this case, the probability P(N) for having N fragments in the system is given by

$$P ( N ) = \int ⅆ {Y}^{N} f ( Γ , N ) = exp ( - Z ) \frac{{Z}^{N}}{N !} . \qquad ( 8 )$$

This is simply the Poisson distribution with parameter Z. In particular, the average number of fragments in the system is given by

$$⟨ N ⟩ = {\sum }_{N = 1}^{∞} NP ( N ) = Z , \qquad ( 9 )$$

which, according to Eq.(7), thus scales exponentially with B.

In fact, more generally, the probability P(n, ΔV) of finding n fragments in any given sub-volume ΔV of configuration space is given by a Poisson distribution:

$$\begin{matrix}\begin{matrix}P ( n , Δ V ) \approx {\sum }_{N = n}^{∞} \frac{N !}{( N - n ) ! n !} {\int }_{Δ V} ⅆ {Y}_{1} … \\ ⅆ {Y}_{n} {\int }_{V} ⅆ {Y}_{n + 1} … ⅆ {Y}_{N} f ( Γ , N ) \\ = \frac{1}{n !} {( exp ( B ) {\int }_{Δ V} \frac{ⅆ Y}{V σ} exp [ - β E ( Y ) ] )}^{n} \frac{1}{Q} {\sum }_{N = n}^{∞} \frac{{Z}^{N - n}}{( N - n ) !} \\ = {z}^{n} / n ! ,\end{matrix} \\ with & ( 10 ) \\ z = exp ( B ) {\int }_{Δ V} \frac{ⅆ Y}{V σ} exp [ - β E ( Y ) ] . & ( 11 )\end{matrix}$$

Finally, the single fragment density is given by

$$\begin{matrix}{f}_{gc} ( Y ) = {\sum }_{N = 1}^{∞} N \int ⅆ {Y}_{2} … \int ⅆ {Y}_{N} f ( Γ = ( Y , {Y}_{2} , … , {Y}_{N} ) , N ) \\ = exp ( - Z ) \frac{1}{V σ} exp [ B - β E ( Y ) ] {\sum }_{N = 1}^{∞} \frac{1}{( N - 1 ) !} {Z}^{( N - 1 )} \\ = \frac{1}{V σ} exp [ B - β E ( Y ) ] ,\end{matrix} \qquad ( 12 )$$

which again scales exponentially with respect to B. Here the subscript “gc” stands for Grand Canonical.

As expected, note that one recovers Eq. (9) for the average number of fragments in the system by integrating f_{gc} over all configurations:

∫dY f_{gc}(Y)=Z. (13)

B. Numerical Method

Equation (12) for the physical single fragment density shows the large dynamical range that may result from the exponential dependence of this quantity with respect to the single fragment-protein potential energy E(Y). This dependence comes from the possible overlap of the non-interacting fragments. This is not an issue in the presence of fragment-fragment interactions, as an upper bound to the fragment density is set by the tightest possible packing of the molecules.

The underlying method developed for the WGCMMC approach to enable the accurate resolution of the above-mentioned dynamical range in densities is presented here.

For numerical purposes, instead of considering a constant B value over the whole system, one may consider a field Bₙᵤₘ(Y) in the single fragment configuration space Y (the subscript “num” standing for numerical). This field can be interpreted as the energy cost for a particle to leave the system specifically from position Y. Instead of Eq. (2), the density of states in this generalized grand canonical ensemble is now given by

$${f}_{num} ( Γ , N ) = \frac{1}{{Q}_{num}} \frac{1}{{V}^{N} {σ}^{N}} \frac{1}{N !} exp [ {\sum }_{i = 1}^{N} {B}_{num} ( {Y}_{i} ) - β U ( Γ , N ) ] , \qquad ( 14 )$$

with the normalization factor (grand partition function) now given by

$${Q}_{num} = {\sum }_{N = 0}^{∞} \frac{1}{N !} \int \frac{ⅆ {Y}^{N}}{{V}^{N} {σ}^{N}} exp [ {\sum }_{i = 1}^{N} {B}_{num} ( {Y}_{i} ) - β U ( Γ , N ) ] . \qquad ( 15 )$$

An analogous derivation as the one used for obtaining Eq. (12) leads to the corresponding single fragment density:

$${f}_{gc , num} ( Y ) = \frac{1}{V σ} exp [ {B}_{num} ( Y ) - β E ( Y ) ] . \qquad ( 16 )$$

Thanks to the field Bₙᵤₘ((Y), one now has a direct handle on the value of the density at each position Y of the single particle configuration space. Thus, by appropriately adapting Bₙᵤₘ(Y) during the convergence phase of the Metropolis Monte Carlo simulation, typically through an iterative process, one may obtain good sampling in all regions of interest. For a Bₙᵤₘ field continuous over Y, this would be achieved by taking

Bₙᵤₘ(Y)≈min(βE(Y)+const, Bₘₐₓ), (17)

leading to similar numerical densities of fragment instances in various regions of space. An upper bound Bₘₐₓ is set on Bₙᵤₘ to avoid unnecessary sampling in strongly unfavorable positions, i.e., essentially for configurations leading to steric clashes. Such an upper bound ensures to preserve the advantages of the Metropolis Monte Carlo scheme over standard Monte Carlo integration algorithms. In practice, the field Bₙᵤₘ(Y) is typically chosen to be independent of the fragment orientation, and to be piece-wise constant on a 3-D grid in x-space (translational-space). Eq. (16) and (17) also show how the purpose of the Bₙᵤₘ(Y) field could have equivalently been achieved by rescaling the single fragment potential energy field E(Y).

Making use of the exponential dependence in B of the density, one can infer the physical fragment density f_{gc}(Y) at any B=B₀=constant value from the simulation results for a given numerical Bₙᵤₘ(Y) field. Assume that one has a sampling {Γᵢ=(Y₁, . . . , Y_{N}_{ᵢ})}_{i=1, . . . , n}_{ₛₙₐₚ} of nₛₙₐₚ snapshots from the numerical distribution fₙᵤₘ(Γ,N). The average of any single fragment quantity A(Y) over the distribution f_{gc}(Y) is then given by

$$\begin{matrix}⟨ A ⟩ = \int ⅆ Y {f}_{gc} ( Y ) A ( Y ) \\ = \int ⅆ Y {f}_{gc , num} ( Y ) \frac{{f}_{gc} ( Y )}{{f}_{gc , num} ( Y )} A ( Y ) \\ ≃ \frac{1}{{n}_{snap}} {\sum }_{i = 1}^{{n}_{snap}} {\sum }_{j = 1}^{{N}_{i}} {w}_{j} A ( {Y}_{j} ) ,\end{matrix} \qquad ( 18 )$$

where wⱼ is the weight assigned to the fragment state Yⱼ, and defined by

$${w}_{j} = \frac{{f}_{gc} ( {Y}_{j} )}{{f}_{gc , num} ( {Y}_{j} )} = exp ( {B}_{0} - {B}_{num} ( {Y}_{j} ) ) . \qquad ( 19 )$$

Results for any B value can thus be inferred from Eqs. (18)-(19). In this way, thanks to the absence of fragment-fragment interactions, simulated annealing of the chemical potential (i.e. variation of B) can be derived analytically given the sampling data for a single Bₙᵤₘ(Y) field.

C. Handling WGCMMC data

The following addresses how the WGCMMC data is to be handled and analyzed.

The starting point for the data interpretation is the relation linking the WGCMMC data to the association constant K_{α} characterizing the binding of the considered fragment to a given region on the protein. This relation for K_{α} is rederived here.

The association constant K_{α} characterizes the equilibrium of the binding process

F+P⇄FP, (20)

and is defined by

$${K}_{a} = \frac{[ FP ]}{[ F ] [ P ]} , \qquad ( 21 )$$

where [P], [F], and [FP] are respectively the concentrations of protein P alone, fragment F alone, and of a particular protein-fragment complex FP (binding mode). The association constant is the basic biologically relevant quantity.

Let us consider a single protein in a volume V. For the sake of the following discussion, take V to be large, although for the actual LMC simulation this need not be the case. The protein concentration is thus given by [P]=1/V. Furthermore, let us note n the average number of fragments in the binding volume ΔV_{b} (in general a volume with limits both in translational and orientational space), and N the average total number of fragments in the system, so that [F]=(N−n)/V and [FP]=n/V. The association constant can thus be written

$${K}_{a} = \frac{n / V}{( N - n ) / V 1 / V} ≃ V \frac{n}{N} \qquad ( 22 )$$

having invoked the thermodynamic limit of large volume V, so that n<<N (N/V→const, for V→∞). The values n and N can be obtained from the fragment density (12):

$$\begin{matrix}n = {\int }_{Δ {V}_{b}}^{} ⅆ Y {f}_{gc} ( Y ) = \frac{{ⅇ}^{B}}{V σ} {\int }_{Δ {V}_{b}}^{} ⅆ Y exp [ - β E ( Y ) ] , & ( 23 ) \\ N = {\int }_{V}^{} ⅆ Y {f}_{gc} ( Y ) = \frac{{ⅇ}^{B}}{V σ} {\int }_{V}^{} ⅆ Y exp [ - β E ( Y ) ] ≃ {ⅇ}^{B} , & ( 24 )\end{matrix}$$

having again invoked the assumption of high protein dilution, so that the total system volume V is much larger than the effective region of interaction between the fragment and the protein, and thus one may consider E(Y)≅0 in deriving the last approximate equality in (24). The association constant now becomes:

$${K}_{a} = \frac{1}{σ} {\int }_{Δ {V}_{b}}^{} ⅆ Y exp [ - β E ( Y ) ] . \qquad ( 25 )$$

On the basis of Eq. (25) one can also write the association constant in terms of the free energy of binding ΔA:

K_{α}=V exp(−βΔA). (26)

where ΔA=A_{FP}−A_{F}, with A_{FP} and A_{F} respectively the free energies of the fragment-protein complex FP and of the fragment F alone:

$$\begin{matrix}{A}_{FP} = - \frac{1}{β} log ( {\int }_{Δ {V}_{b}}^{} ⅆ Y exp [ - β E ( Y ) ] ) , & ( 27 ) \\ {A}_{F} = - \frac{1}{β} log {\int }_{V}^{} ⅆ Y = - \frac{1}{β} log ( V σ ) . & ( 28 )\end{matrix}$$

The critical value B_{c} that is associated to the binding volume ΔV_{b} is defined as the value for which the average number of fragments in the binding site is one. From Eq. (23) follows:

$$\begin{matrix}n ( {B}_{c} ) = 1 & ⇔ & {ⅇ}^{- {B}_{c}} = \frac{1}{V σ} {\int }_{Δ {V}_{b}}^{} ⅆ Y exp [ - β E ( Y ) ] ,\end{matrix} \qquad ( 29 )$$

and from (25), (26) and (29) one sees that B_{c} is directly related to Kₐ and ΔA as follows:

$$\begin{matrix}{K}_{a} = V {ⅇ}^{- {B}_{c}} , & ( 30 ) \\ Δ A = \frac{1}{β} {B}_{c} . & ( 31 )\end{matrix}$$

Thus, a low B_{c} value reflects a high affinity binding mode, and inversely a high B_{c} value reflects a low affinity mode.

The critical value B_{c} can be computed from the WGCMMC data using definition (29), as well as Eqs (18) and (19):

$$\begin{matrix}1 = n ( {B}_{c} ) \\ = {\int }_{Δ {V}_{b}} ⅆ Y {f}_{gc} ( Y ) ≅ \frac{1}{{n}_{snap}} {\sum }_{i = 1}^{{n}_{snap}} {\sum }_{frag j ∈ Δ {V}_{b}} exp [ {B}_{c} - {B}_{num} ( {Y}_{j} ) ] ⇔ \\ {B}_{c} = - log ( \frac{1}{{n}_{snap}} {\sum }_{i = 1}^{{n}_{snap}} {\sum }_{frag j ∈ Δ {V}_{b}} exp [ - {B}_{num} ( {Y}_{j} ) ] ) .\end{matrix} \qquad ( 32 )$$

Equations (30), (31) and (32) provide the basic relations for interpreting the WGCMMC data.

Binding Analysis

A first estimate of the binding affinity of a given fragment for different regions on the protein surface can be obtained by assigning a critical B_{c} to each fragment-residue pair. These B_{c} values are obtained from the WGCMMC data by applying relation (32), and by assigning a binding volume ΔV_{b} to each residue based of the following proximity criteria: A fragment state is considered to be in proximity of a given residue if at least one fragment-protein atom pair (a, b) is such that

r_{ab}<a (R_{VdW,a}+R_{vdw,b}), (33)

where r_{ab} is the distance between the two atoms, R_{VdW} is the Van der Walls radii, and α is a numerical parameter (typically α=1.2). The Van der Walls radii are typically defined as half the Lennard-Jones parameter from the considered molecular-mechanics force-field (e.g. AMBER) used for the Monte Carlo simulation.

The volume defined on the basis of the proximity criteria is in general only a crude estimate of a binding mode volume. The corresponding B_{c} values must therefore be interpreted accordingly. Nonetheless, comparing sets of B_{c} values obtained in this way for different fragment types has proven valuable to help identify protein binding sites as follows: A binding site is identified as a set of neighboring residues with low B_{c} values (high affinity) for multiple fragments with different physico-chemical properties. This approach is based on the assumption that diverse interactions in a localized region are the necessary condition for ensuring the specificity of a binding site. This numerical identification of binding sites is preferably complemented by experimental binding information, such as co-crystal X-ray data and mutational analysis.

Compared to the above described residue-based proximity criteria, more detailed calculations of the binding mode volumes ΔV_{b} are necessary to provide more accurate estimates of the free energy of binding using Eq. (32). Such improved binding mode volume estimates are determined by identifying “humps” in the fragment distribution. This can be achieved by clustering sampled fragment states belonging to a same potential energy well. For this purpose one makes use of the potential energies saved for the sampled fragment states.

Chemistry Design

With the purpose of data reduction, the LCD chemistry design software clumps the sampled fragment instances together. Clumping in LCD is usually carried out at a relatively fine-grained level, so that the clumping volume AVC (limited both in translational and orientational space) is different from a true binding mode volume ΔV_{b} of the fragment. In fact, a binding mode volume is usually composed of many clump volumes. Each clump is thus assigned the B_{c} value of the binding mode volume to which it belongs.

Using the WGCMMC-type data, average clump positions x_{c} and quaternion representation q_{c} of average clump orientation can be computed by the following weighted averages:

$$\begin{matrix}⟨ {x}_{c} ⟩ = \frac{{\sum }_{i} {w}_{i} {x}_{i}}{{\sum }_{i} {w}_{i}} , & ( 37 ) \\ ⟨ {q}_{c} ⟩ = {\sum }_{i} {w}_{i} {q}_{i} -> Normalize {q}_{c} , & ( 38 )\end{matrix}$$

where the sums are over all fragments i in the clump.

Within the chosen protein binding site, clumps of different fragment types can then be assembled into actual candidate drug leads, usually composed of four to five fragments. Assembly of fragments is carried out based on binding affinity of the different fragments (B_{c} values), and on geometric proximity using a variety of rules by which organic fragments may bond together, as is well known in the art.

D. Process Implementation

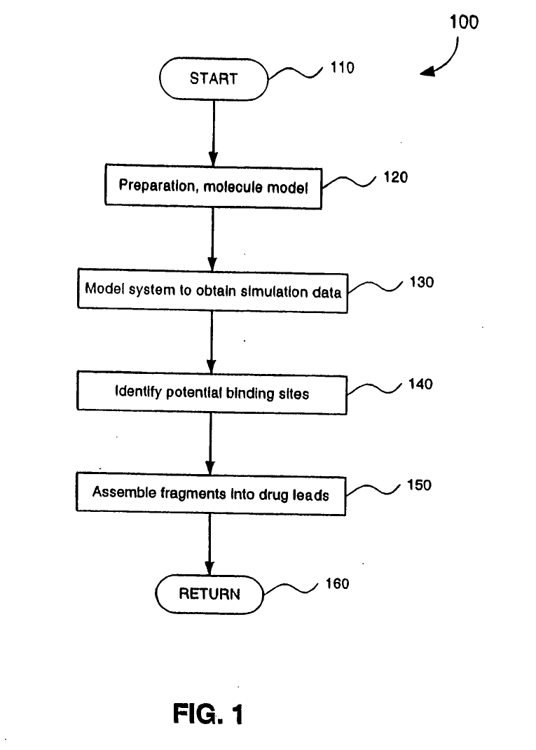

In light of the above analytical description of WGCMMC processing, its logic can be implemented in the broader simulation context as illustrated in FIG. 1, according to an embodiment of the invention. The overall process starts at step 110. In step 120, a model is constructed for the molecules to be simulated, i.e., a protein as well as different types of rigid molecular fragments whose interaction with the protein will be analyzed. In step 130, the thermodynamic equilibrium of the system is modeled so that the interactions between a given fragment type and the protein at thermodynamic equilibrium can be understood. This step results in simulation data that includes, for each fragment state, the fragment's position, orientation, weight, and fragment-protein energy. Step 130 is carried out for each fragment type of interest. These simulations for the different fragment types are performed independently from one another and can therefore be conveniently carried out simultaneously on a cluster of computers, without any communication required between processes. In step 140, potential binding sites are identified on the protein. In step 150, fragments are assembled into drug leads. The overall process concludes at step 160. Each of these steps is described in greater detail below.

Molecule Preparation

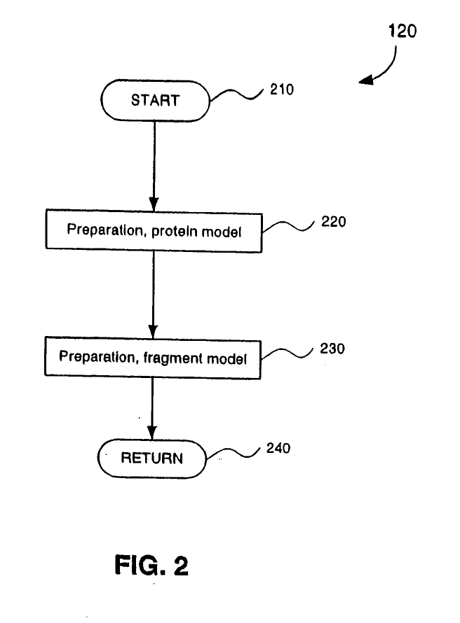

Step 120, the preparation of the molecular model, is illustrated in FIG. 2. This process starts at step 210. Protein preparation takes place in step 220. A protein can be viewed as a biological macro-molecule to which a prospective ligand binds. The basic protein structure is provided by experimental X-ray crystallography data, typically downloaded from a data base [e.g. the from the Protein Data Bank (PDB), Research Collaboratory for Structural Bioinformatics (RCSB), Rutgers Univ., NJ]. If required, the protein structure is completed for missing substructures, which in some cases may be a limited number of heavy atoms or, in other cases, entire segments of an amino-acid chain. Hydrogen atoms, not resolved by X-ray crystallography, are added as well. Conformer and protonation state issues for the amino-acids HIS, ASP, GLU, CYS, TYR, THR, and SER are also resolved at this stage. Such a process for protein preparation is disclosed and claimed in a co-pending U.S. patent application Ser. No. 60/450,711, filed on Mar. 3, 2003, and incorporated herein by reference in its entirety.

Fragment preparation takes place in step 230. The structure and partial charges of the small organic fragments are completed with an ab initio, i.e. quantum mechanical based, code. This calculation is typically carried out in the framework of the Density Functional Theory (DFT) approximation using the code Gaussian (M. J. Fish et.al., “Gaussian 98, revision A.9,” 1998. Gaussian Inc., Pittsburgh, Pa.). This step also assigns the atom types from the molecular mechanics force-field (e.g. AMBER) applied in the subsequent Monte Carlo simulation. The process concludes at step 240.

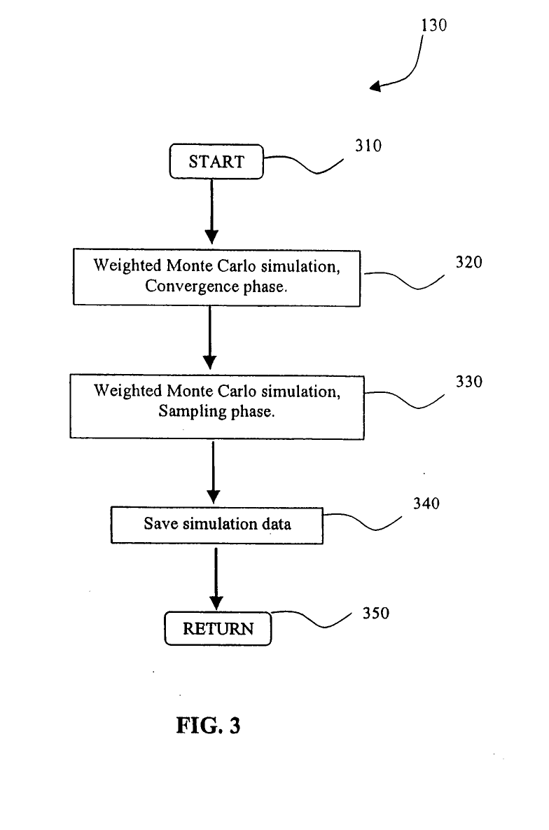

The step of modeling the thermodynamic system of the protein-fragment interaction is illustrated in greater detail in FIG. 3, according to an embodiment of the invention. The process starts at step 310. In step 320, a convergence phase of the weighted grand canonical Metropolis Monte Carlo simulation is executed. This is followed by a sampling phase in step 330. Steps 320 and 330 are described in greater detail below. The resulting simulation data is saved in step 340. The process concludes in step 350.

Convergence Phase of LMC Simulation

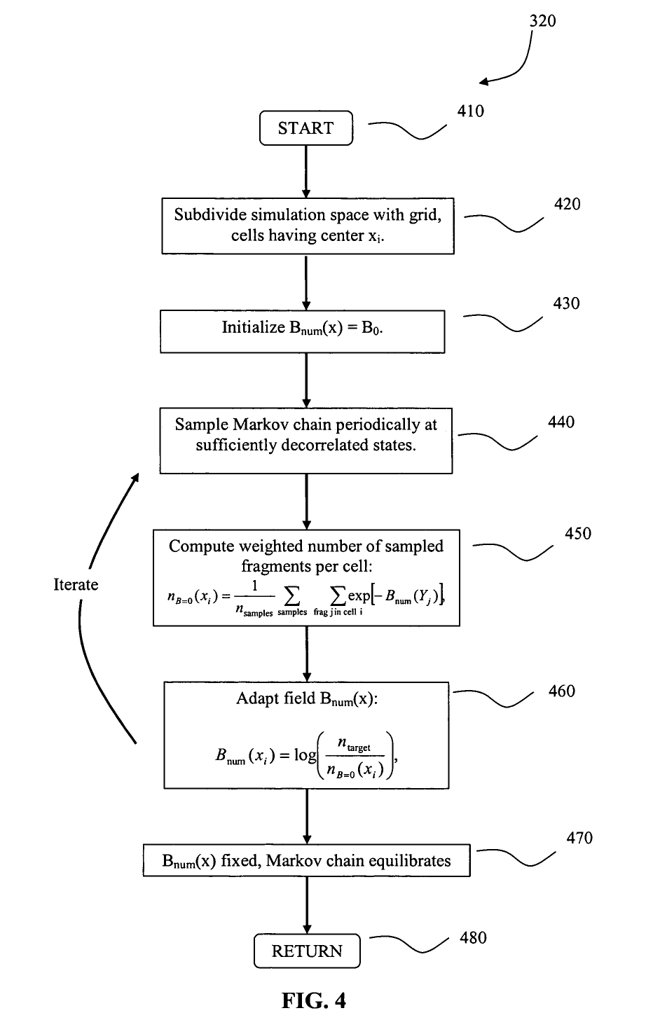

Step 320, the convergence phase of the LMC simulation, is illustrated in FIG. 4. In this first stage of the LMC simulation, the numerical B-field, Bₙᵤₘ, and the Markov chain generated by the LMC stepping are converged.

The process starts with step 410. In step 420, the simulation space is subdivided with a grid. Typically, the 3-dimensional translational space of the simulation system is subdivided by an orthogonal, equidistant grid, with centers xᵢ. Grid size is based on the variation scale of the interaction force-field, typically of the order of one Angstrom. The Bₙᵤₘ field is then initialized on this grid to a constant value Bo (Bₙᵤₘ≡Bo), as shown in step 430.

Stepping of the system state is then carried out using the Metropolis Monte Carlo scheme for grand canonical simulations [Adams, D. J., Molecular Physics 29:307-311 (1975); Mezei, M., Molecular Physics 61:565-582 (1987)]. (These references are incorporated herein by reference in their entireties.) At regular intervals in the stepping of the convergence phase, sufficiently long to ensure decorrelation of states, the fragment distributions are saved, as shown in 440.

With progressively improved statistics, the fragment distributions are then monitored periodically as given in step 450, where the weighted number of sampled fragments in each grid cell xᵢ is computed as follows:

$${n}_{B = 0} ( {x}_{i} ) = \frac{1}{{n}_{samples}} {\sum }_{samples} {\sum }_{frag j i n cell i} exp [ - {B}_{num} ( {Y}_{j} ) ] , \qquad ( 40 )$$

where nₛₐₘₚₗₑₛ is the number of samples saved up to the current point in the convergence phase. Equation (40) is an application of Eqs. (18)-(19) for B₀=0.

Based on these statistics, the field Bₙᵤₘ(x) is then adapted in step 460, by making use of the exponential dependence in Bₙᵤₘ(x) of the number of fragments in each grid cell i. In this way, each cell is assigned a constant value Bₙᵤₘ(xᵢ) as follows:

$${B}_{num} ( {x}_{i} ) = log ( \frac{{n}_{target}}{{n}_{B = 0} ( {x}_{i} )} ) , \qquad ( 41 )$$

the goal being to achieve a similar average number of sampled fragments n_{target} within all cells. An upper bound Bₘₐₓ is set on Bₙᵤₘ to avoid spending too much computing time on sampling very unfavorable positions, i.e., mainly for configurations leading to steric clashes or for fragment states far away from the protein surface where the binding interaction is low. In this way one still ensures the numerical advantages of the Metropolis Monte Carlo scheme over basic Monte Carlo integration algorithms.

Adapting the field Bₙᵤₘ(X) is an iterative process of steps 440 to 460. Indeed, the first Bₙᵤₘ updates are based on some very non-uniform sampling, thorough in deep energy pockets, but poor in shallow ones. As the Bₙᵤₘ(x) field is adapted, the sampling is globally improved and the adjustment of Bₙᵤₘ(x) can be further refined.

In step 470 of the convergence phase, the Bₙᵤₘ(x) field is finally kept fixed, which enables the Markov chain to fully equilibrate.

The acceptance probabilities for the various types of Monte Carlo steps in the framework of the grand canonical ensemble with spatially varying Bₙᵤₘ(x) field are as follows:—

Moving a fragment within the simulation system: Assuming symmetric attempts, moving a fragment from position Yₐ=(xₐ, Ωₐ) to position Y_{b}=(x_{b}, Ω_{b}) is accepted with probability:

acc(Yₐ→Y_{b})=min(1, α), (42)

with α=exp([B(x_{b})−B(xₐ)]−β[E(Y_{b})−E(Yₐ)]). (43)

Inserting a fragment into the simulation system: Assuming no biased sampling, and considering that N fragments are already present in the system, the probability of accepting the insertion of a fragment at position Y=(x, Ω) is given by:

$$\begin{matrix}acc ( N -> N + 1 ) = min ( 1 , α ) , \\ with & ( 44 ) \\ α = \frac{1}{N + 1} exp ( B ( x ) - β E ( Y ) ) . & ( 45 )\end{matrix}$$

Deleting a fragment from the simulation system: The probability of deleting a fragment at position Y=(x, Ω), assuming that N+1 fragments are initially in the system, is given by:

acc(N+1→N)=min(1, α), (46)

with α=(N+1)exp(−B(x)+βE(Y)). (47)

Equations (42) to (47) can naturally be generalized to various types of biased sampling, such as preferential sampling or cavity bias.

Sampling Phase of MC Simulation

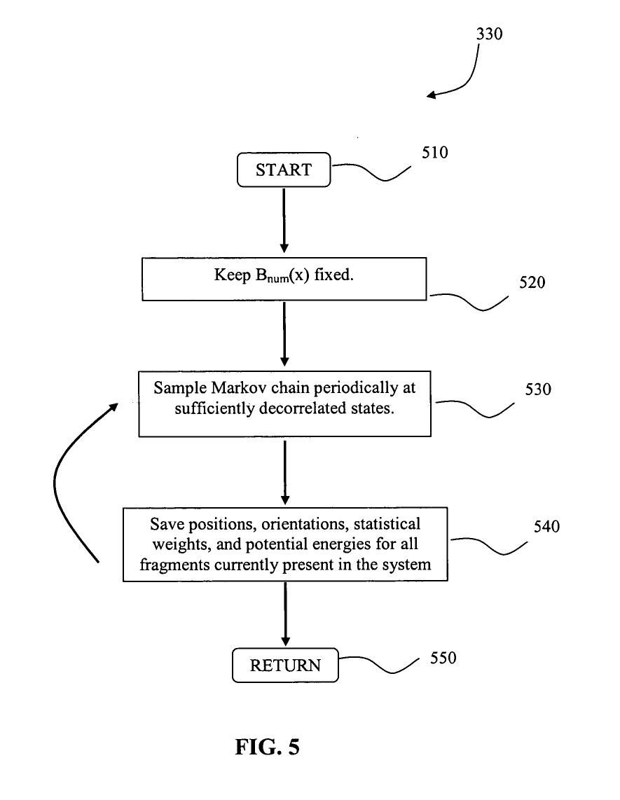

The numerical B-field, Bₙᵤₘ, is kept fixed throughout the second stage, the so-called sampling phase, of the MC simulation. This phase, step 330 of FIG. 3, is illustrated in greater detail in FIG. 5. The process starts with step 510. In step 520, Bₙᵤₘ(x) is further kept fixed. In step 530, the equilibrated Markov chain is sampled periodically at sufficiently decorrelated states until a statistically appropriate amount of sampling data is acquired. As shown in step 540, saving the state of the system consists of storing the positions x, orientations Ω, weights w=exp(−Bₙᵤₘ(x)), and fragment-protein potential energies E(Y) of all fragments currently present in the system. The sampling process concludes at step 550.

Identifying Binding Modes

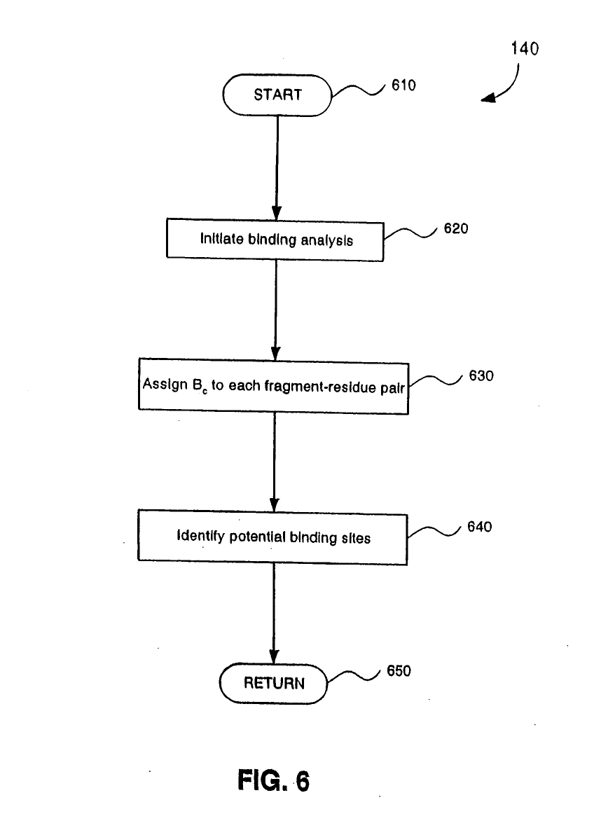

FIG. 6 illustrates the process of identifying potential binding sites, according to an embodiment of the invention. The process starts with step 610. In step 620, logic such as the Locus Binding Analysis (LBA) software package begins execution. In step 630, a value B_{c} is assigned to each fragment-residue pair. In step 640, potential binding sites are identified on the basis of the B_{c} values. As discussed above, these B_{c} values are obtained from the WGCMMC data by applying relation (32), where the volume ΔV_{b} is defined for each residue on the basis of the proximity criteria. Recall from Eq. (33) above that a fragment is considered to be in proximity of a given residue if at least one fragment-protein atom pair (a, b) is such that

r_{ab}<α(R_{VdW,a}+R_{VdW,b}), (33)

where r_{ab} is the distance between the two atoms, R_{VdW} is the Van der Walls radii, and α is a numerical parameter (typically α=1.2). The Van der Walls radii are typically defined as half the Lennard-Jones parameter from the considered molecular-mechanics force-field (e.g. AMBER) used for the Monte Carlo simulation. A binding site is then identified as a set of residues with low B_{c} values (high affinity) for multiple fragment types with diverse physico-chemical properties. The process concludes at step 650.

Assembling Fragments in the Binding Site

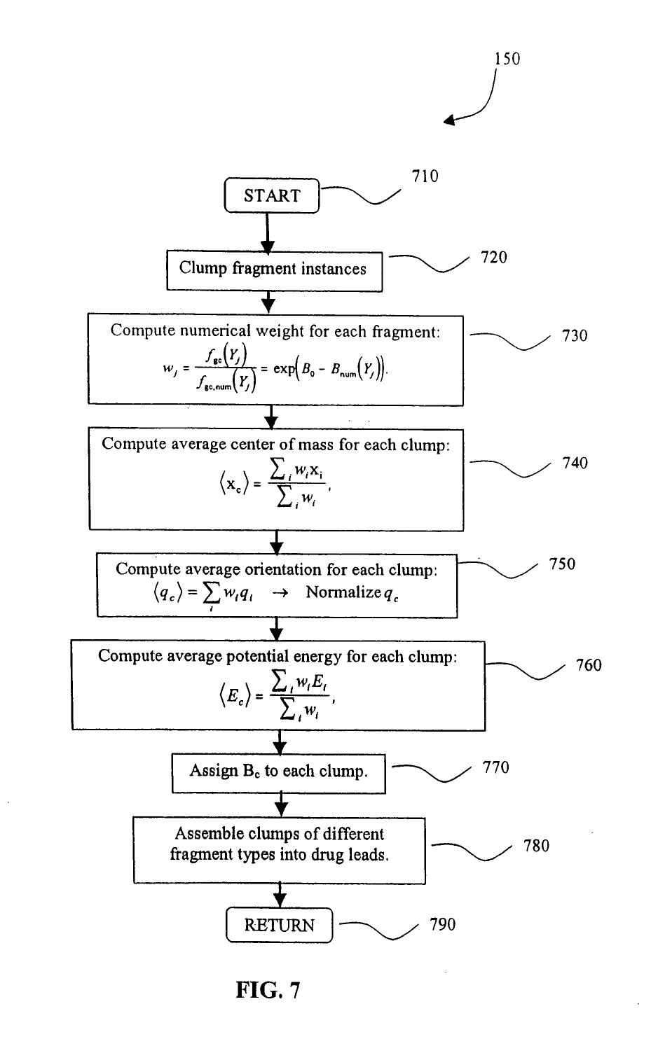

Step 150 of FIG. 1, the step of assembling fragments into drug leads, is illustrated in greater detail in FIG. 7, according to an embodiment of the invention. The process starts with step 710. With the purpose of data reduction, fragment instances are clumped together in step 720. Clumping is carried out at a relatively fine-grained level (both in translational and orientational space), so that the clumping volume ΔV_{c} is different from a true binding volume. In fact, a binding mode volume is usually composed of many clump volumes. The purpose of this clumping is to achieve some level of data reduction before carrying on with the fragment assembly into drug leads. From a combinatorial point of view, this assembly indeed becomes increasingly complex and therefore computationally intensive with increasing number of considered fragment poses.

In steps 730 through 750, weighted average clump positions x_{c}, and quaternion representation of weighted average clump orientation q_{c} is computed as described earlier:

$$\begin{matrix}⟨ {x}_{c} ⟩ = \frac{{\sum }_{i} {w}_{i} {x}_{i}}{{\sum }_{i} {w}_{i}} , & ( 37 ) \\ ⟨ {q}_{c} ⟩ = {\sum }_{i} {w}_{i} {q}_{i} -> Normalize {q}_{c} , & ( 38 )\end{matrix}$$

where the sums are over all fragments i in the clump.

In the same way, as appears in step 760, one may also compute the average potential energy of the clump:

$$⟨ {E}_{c} ⟩ = \frac{{\sum }_{i} {w}_{i} {E}_{i}}{{\sum }_{i} {w}_{i}} , \qquad ( 39 )$$

where Eᵢ is the potential energy of interaction of fragment i with the protein.

In step 770, each clump is assigned the B_{c} value of the binding mode volume to which it belongs.

In step 780, within the chosen protein binding site, clumps of different fragment types are then assembled into actual candidate drug leads, usually (though not always) composed of four to five fragments. Assembly of fragments is carried out based on binding affinity of the different fragments (B_{c} values), and on geometric proximity, using a variety of rules by which organic fragments may bond together as is well known in the art.

III. Computing Environment

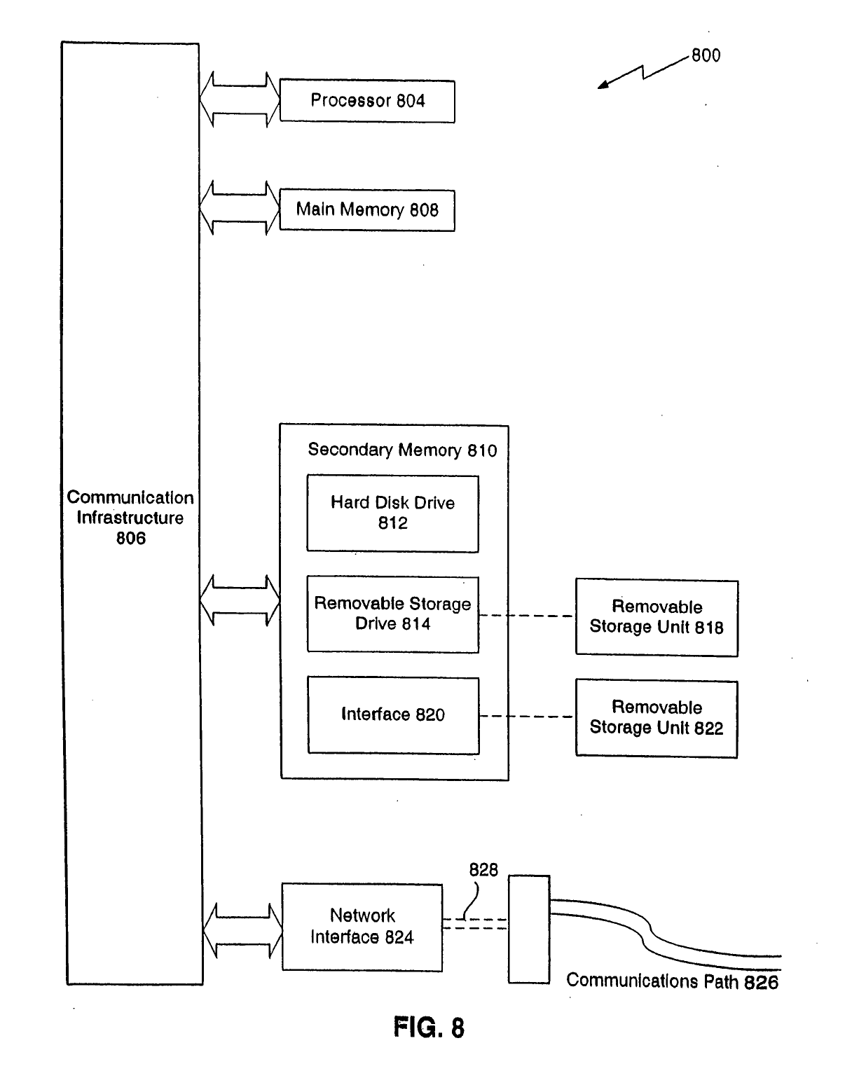

The present invention may be implemented using software and may be implemented in conjunction with a computing system or other processing system. An example of such a computer system 800 is shown in FIG. 8. The computer system 800 includes one or more processors, such as processor 804. It is to be noted that the here-described fragment-based computation is particularly well suited for being carried out on a computer cluster, each cluster node computing the interaction of a given fragment type with the target protein. The processor 804 is connected to a communication infrastructure 806, such as a bus or network. Various software implementations are described in terms of this exemplary computer system. After reading this description, it will become apparent to a person skilled in the relevant art how to implement the invention using other computer systems and/or computer architectures.

Computer system 800 also includes a main memory 808, preferably random access memory (RAM), and may also include a secondary memory 810. The secondary memory 810 may include, for example, a hard disk drive 812 and/or a removable storage drive 814, representing a magnetic tape drive, an optical disk drive, etc. The removable storage drive 814 reads from and/or writes to a removable storage unit 818 in a well-known manner. Removable storage unit 818 represents a magnetic tape, optical disk, or other storage medium that is read by and written to by removable storage drive 814. As will be appreciated, the removable storage unit 818 can include a computer usable storage medium having stored therein computer software and/or data.

In alternative implementations, secondary memory 810 may include other means for allowing computer programs or other instructions to be loaded into computer system 800. Such means may include, for example, a removable storage unit 822 and an interface 820. An example of such means may include a removable memory chip (such as an EPROM, or PROM) and associated socket, or other removable storage units 822 and interfaces 820 which allow software and data to be transferred from the removable storage unit 822 to computer system 800.

Computer system 800 may also include one or more communications interfaces, such as network interface 824. Network interface 824 allows software and data to be transferred between computer system 800 and external devices. Examples of network interface 824 may include a modem, a network interface (such as an Ethernet card), a communications port, a PCMCIA slot and card, etc. Software and data transferred via network interface 824 are in the form of signals 828 which may be electronic, electromagnetic, optical or other signals capable of being received by network interface 824. These signals 828 are provided to network interface 824 via a communications path (i.e., channel) 826. This channel 826 carries signals 828 and may be implemented using wire or cable, fiber optics, an RF link and other communications channels.

In this document, the terms “computer program medium” and “computer usable medium” are used to generally refer to media such as removable storage units 818 and 822, a hard disk installed in hard disk drive 812, and signals 828. These computer program products are means for providing software to computer system 800.

Computer programs (also called computer control logic) are stored in main memory 808 and/or secondary memory 810. Computer programs may also be received via communications interface 824. Such computer programs, when executed, enable the computer system 800 to implement the present invention as discussed herein. In particular, the computer programs, when executed, enable the processor 804 to implement the present invention. Accordingly, such computer programs represent controllers of the computer system 800. Where the invention is implemented using software, the software may be stored in a computer program product and loaded into computer system 800 using removable storage drive 814, hard drive 812 or communications interface 824.

### IV. CONCLUSION

While various embodiments of the present invention have been described above, it should be understood that they have been presented by way of example, and not limitation. It will be apparent to persons skilled in the relevant art that various changes in detail can be made therein without departing from the spirit and scope of the invention. Thus the present invention should not be limited by any of the above-described exemplary embodiments.

What is claimed is:

1. A method for modeling a system that includes a protein and a plurality of fragment types in order to identify drug leads, the method comprising:
initiating a weighted grand canonical Metropolis Monte Carlo simulation of the system;
subdividing the space of the simulation system with a grid, with xᵢ the centers of the grid cells;
initializing a numerical chemical potential field Bₙᵤₘ=B₀ on the grid;
periodically sampling the Markov chain associated with the Metropolis Monte Carlo simulation, so as to compute the weighted number of sampled fragments per cell:
$${n}_{B = 0} ( {x}_{i} ) = \frac{1}{{n}_{samples}} {\sum }_{samples} {\sum }_{frag j i n cell i} exp [ - {B}_{num} ( {Y}_{j} ) ] ;$$
iteratively adapting the field Bₙᵤₘ(x) according to
$${B}_{num} ( {x}_{i} ) = log ( \frac{{n}_{target}}{{n}_{B = 0}} ) ,$$
fixing the field Bₙᵤₘ(X) such that the Markov chain associated with the Metropolis Monte Carlo simulation equilibrates; and
outputting samples from the equilibrated Markov chain.

2. The method of claim 1, further comprising:
sampling the Markov chain periodically, with sufficiently long interspacing to ensure decorrelated states of the system; and
saving positions, orientations, fragment-protein potential energies, and statistical weights for all fragments present in a current state of the system.

3. The method of claim 2, further comprising:
performing binding analysis of the system, based on the positions, orientations, fragment-protein potential energies, and statistical weights for all fragment states provided by the sampling.

4. The method of claim 3, wherein said performing step comprises:
i) making use of the properties of the grand canonical ensemble to estimate the binding affinity of the fragment for different regions of the protein surface by assigning a critical value B_{c} to each fragment-residue pair, using the positions, orientations, and statistical weights for all fragment states provided by the sampling; and
ii) identifying potential binding sites on the protein based on the B_{c} values.

5. The method of claim 2, further comprising:
assembling the fragments into drug leads for a considered binding site, based on binding affinity of the fragment types (B_{c} values) for the considered binding site, and on geometric proximity using rules by which organic fragments may bond together.

6. A computer program product comprising a computer usable medium having computer readable program code that enables a computer to model a system that comprises a protein and a plurality of fragments in order to identify drug leads, the computer program product comprising:
first computer readable program code that initiates a weighted grand canonical Metropolis Monte Carlo simulation;
second computer readable program code that causes the computer to subdivide the space of the simulation system with a grid, with xᵢ the centers of the grid cells;
third computer readable program code that causes the computer to initialize a field Bₙᵤₘ(xᵢ)=B₀;
fourth computer readable program code that causes the computer to compute the weighted number of sampled fragments per cell,
$${n}_{B = 0} ( {x}_{i} ) = \frac{1}{{n}_{samples}} {\sum }_{samples} {\sum }_{frag j i n cell i} exp [ - {B}_{num} ( {Y}_{j} ) ] ,$$
fifth computer readable program code that causes the computer to iteratively adapt the field Bₙᵤₘ(x) according to
$${B}_{num} ( {x}_{i} ) = log ( \frac{{n}_{target}}{{n}_{B = 0}} ) ,$$
sixth computer readable program code that causes the computer to keep the field Bₙᵤₘ(x) fixed, so that the Markov chain associated with the Metropolis Monte Carlo scheme can equilibrate; and
seventh computer readable program code that causes the computer to output samples from the equilibrated Markov chain.

7. The computer program product of claim 6, further comprising:
seventh computer readable program code that causes the computer to sample the Markhov chain periodically at sufficiently decorrelated states of the system; and
eighth computer readable program code that causes the computer to obtain positions, orientations, fragment-protein potential energies, and statistical weights for all fragments present in a current state of the system.

8. The computer program product of claim 7, further comprising:
ninth computer readable program code that causes the computer to perform binding analysis based on the positions, orientations, and statistical weights for all fragments at each sampled state of the system.

9. The computer program product of claim 8, wherein said ninth computer readable program code comprises:
computer readable program code that causes the computer to assign a critical value B_{c} to each fragment-residue pair based on the positions, orientations, and statistical weights for all fragments at each state; and
computer readable program code that causes the computer to identify potential binding sites on the protein based on the B_{c} values.

10. The computer program product of claim 8, further comprising:
tenth computer readable program code that causes the computer to assemble the fragments into drug leads for a considered binding site based on binding affinity of the fragment types (B_{c} values), and on geometric proximity using rules by which organic fragments may bond together.

11. A system for modeling a system that includes a protein and a plurality of different fragment types in order to identify drug leads, the system comprising:
A. means for initiating a weighted grand canonical Metropolis Monte Carlo simulation of the system;
B. means for subdividing the space of the simulation system with a grid, with x_{i the centers of the grid cells;}
C. means for initializing a numerical chemical potential field Bₙᵤₘ=B₀ on the grid;
D. means for computing the weighted number of sampled fragments per cell,
$${n}_{B = 0} ( {x}_{i} ) = \frac{1}{{n}_{samples}} {\sum }_{samples} {\sum }_{frag j in cell i} exp [ - {B}_{num} ( {Y}_{j} ) ] ;$$
E. means for iteratively adapting the field Bₙᵤₘ(X) such that
$${B}_{num} ( {x}_{i} ) = log ( \frac{{n}_{target}}{{n}_{B = 0}} ) ,$$
F. means for fixing the field Bₙᵤₘ(x) such that the associated Markhov chain equilibrates; and
G. means for outputting samples from an equilibrated Markov chain.
